(19) United States
(12) Patent Application Publication
(10) Pub. No.: US 2012/0184925 A1
(43) Pub. Date: Jul. 19, 2012

(54) MULTIPLE SEPTUM PORT

(75) Inventor: Kerry GRANT, Northbridge, MA (US)

(73) Assignee: Navilyst Medical, Inc., Marlborough, MA (US)

(21) Appl. No.: 13/009,466

(22) Filed: Jan. 19, 2011

(51) Int. Cl. A61M 5/00

(52) U.S. Cl. 604/288.01

(57) ABSTRACT

A multiple septum vascular access port is provided, including a housing, an inner and outer wall, two reservoirs, two septa, and two fluid lines in fluid communication with the two reservoirs. The reservoirs and septa are arranged so that one reservoir and septum is nested within the other reservoir and septum.

### FIELD OF THE INVENTION

The present invention relates to catheters for use in medical applications wherein fluids are repeatedly infused into or sampled from the vascular system and, more specifically, to multiple-septum vascular access ports for use with such catheters.

### BACKGROUND OF THE INVENTION

Certain medical treatments require implantation of vascular access ports (“ports”), through which fluids may be injected or removed repeatedly over time, via catheter, from the vascular system. A port is made up of (i) a housing, which defines a reservoir, (ii) a septum, which covers the reservoir and can be punctured by a needle for fluid input or removal, and which typically seals itself once the needle is withdrawn, and (iii) a fluid path through which fluid can pass between the reservoir and a catheter attached to the port. A port will usually be connected to a vascular catheter, which catheter is inserted into the vascular system so that its tip lies in a central vascular location such as the vena cava. A port is typically implanted subcutaneously and, because it cannot be seen clearly through the skin, may have ridges or markings that permit location of the septum by palpation or by visualization under a fluoroscope.

In instances where it is desirable to have more than one fluid path through which to inject or remove fluid, a multiple septum port may be utilized, in which multiple reservoirs and septa are arranged alongside one-another within a single housing to keep the fluid paths for each injected fluid separate. A potential limitation of this side-by-side design is that ports having more than one septum may be significantly bulkier than ports with a single septum. It would be desirable to reduce the bulk of multiple septum ports as a means of improving patient comfort and decreasing the unsightliness of the implanted port.

### SUMMARY OF THE INVENTION

In one aspect, the present invention provides a multiple septum port having a reduced bulk and profile by locating one reservoir and septum within a second reservoir and septum, and separating the two reservoirs and septa with a wall. In one embodiment, the present invention includes a multiple septum port made up of a housing having an inner wall and an outer wall separated by a space, a first reservoir defined by the inner wall, and a second reservoir surrounding the first reservoir which is defined by the space between the first and second walls. The first reservoir is overlaid by a first septum and is in fluid communication with a first fluid line, while the second reservoir is overlaid by a second septum and is in fluid communication with a second fluid line. When either septum is penetrated, fluid can flow into or out of the underlying reservoir and the attached fluid line.

In certain embodiments, each of the inner and outer wall is characterized by a height. In some embodiments, the height of the inner wall is greater than that of the outer wall. In other embodiments, the height of the inner wall is less than or equal to that of the outer wall.

In certain embodiments, the first reservoir is located concentrically inside of the second reservoir.

In certain embodiments, the first fluid line is located, for part of its length, inside of the second fluid line, so that it can be connected to a catheter which in turn runs within another catheter that is connected to the second fluid line. In other embodiments the first fluid line extends, for at least part of its length, beneath the second reservoir. In still other embodiments, the first fluid line extends, for at least part of its length, through the second reservoir.

In certain embodiments, each of the first and second reservoirs is characterized by a depth. In one embodiment, the first reservoir has a depth greater than that of the second reservoir. In other embodiments, the first reservoir has a depth less than or equal to that of the second reservoir.

In another aspect, the present invention relates to a method of treating a patient using a multiple septum vascular access port by implanting the port subcutaneously into a patient and palpating the inner wall of the device or visualizing radiopaque markings on the inner wall or outer wall of the device under fluoroscopy to facilitate insertion of a needle or needles into the first or second septa and infusing fluids into or sampling fluids from the vascular system.

### BRIEF DESCRIPTION OF THE DRAWINGS

In the drawings, like reference characters generally refer to the same parts throughout the different views. Drawings are not necessarily to scale, as emphasis is placed on illustration of the principles of the invention.

FIGS. 1(a) and 1(b) are a schematic top view and a cross-sectional view from the top, respectively, of a port device in accordance with an embodiment of the present invention.

FIG. 2 is a schematic side view of a port device in accordance with an embodiment of the present invention.

FIG. 3 is a schematic cross-sectional view of a port device in accordance with an embodiment of the present invention.

FIG. 4 is a schematic view of three alternative arrangements of first and second fluid lines as they exit the housing of a port device in accordance with embodiments of the present invention.

FIGS. 5(a) and 5(b) are cross-sectional views of alternate embodiments of the present invention having different reservoir depths.

FIGS. 6(a) and 6(b) are cross-sectional views of alternate embodiments of the present invention having different wall heights.

FIGS. 7(a) and 7(b) are cross-sectional views of alternate embodiments of the present invention having different fluid line arrangements.

### DETAILED DESCRIPTION OF THE PREFERRED EMBODIMENTS

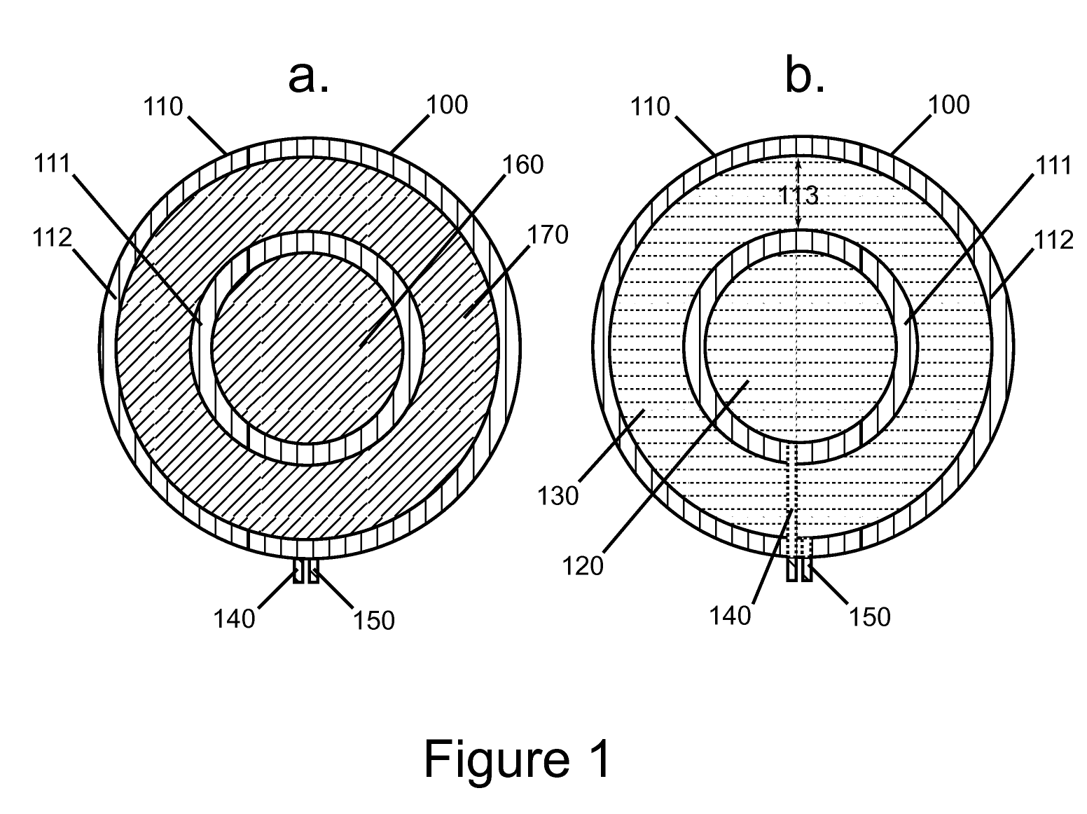

FIGS. 1-7 illustrate several embodiments of the present invention. Port 100 is a multiple septum vascular access port with a housing 110 that includes inner and outer walls 111, 112 having a distance therebetween 113, which walls define first and second reservoirs 120, 130. Each of the first and second reservoirs 120, 130 are topped by first and second septa, 160, 170, respectively. The first and second fluid lines, 140, 150, are in fluid communication with the first and second reservoirs, 120, 130, respectively.

The shape and orientation of inner and outer walls 111, 112, will determine the shape of the reservoirs 120, 130 and the septa 160, 170. In the embodiment shown in FIGS. 1-3, outer wall 112 and inner wall 111 are circular and concentric, such that the first reservoir 120 and the second reservoir 130, and the first septum 160 and the second septum 170 are arranged concentrically about the center of the circle defined by the outer wall 112, and the space 113 between the inner and outer walls 111, 112 is constant throughout port 100. In alternate embodiments, the inner and outer walls 111, 112 are in any suitable non-circular configuration, resulting in non-circular reservoirs 120, 130 and septa 160, 170. In still other embodiments, the inner wall 111 will not be positioned concentrically within the outer wall 112, such that the first reservoir 120 and the first septum 160 do not sit centrally within the second reservoir 130 and the second septum 170, respectively, and the space 113 between inner and outer walls 111, 112 is smaller in some areas of the port 100 than in others.

Septa 160, 170 can be made of any suitable self-sealing material such as silicone, and may be of any suitable thickness with any suitable surface shape or other physical characteristic.

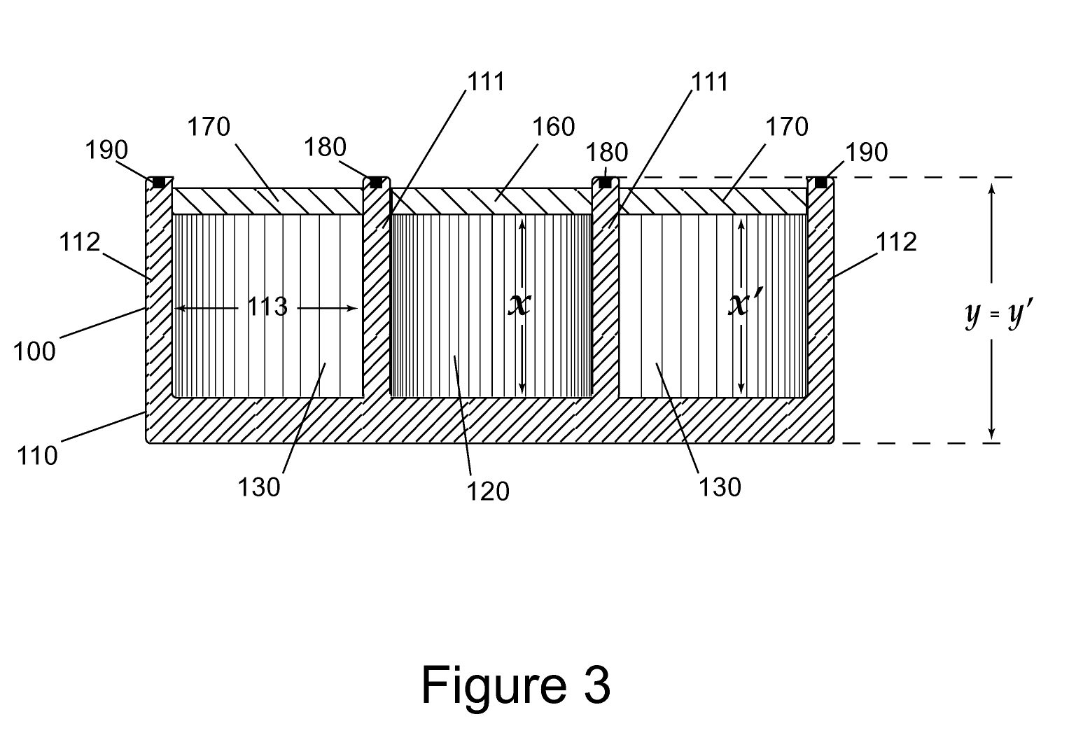

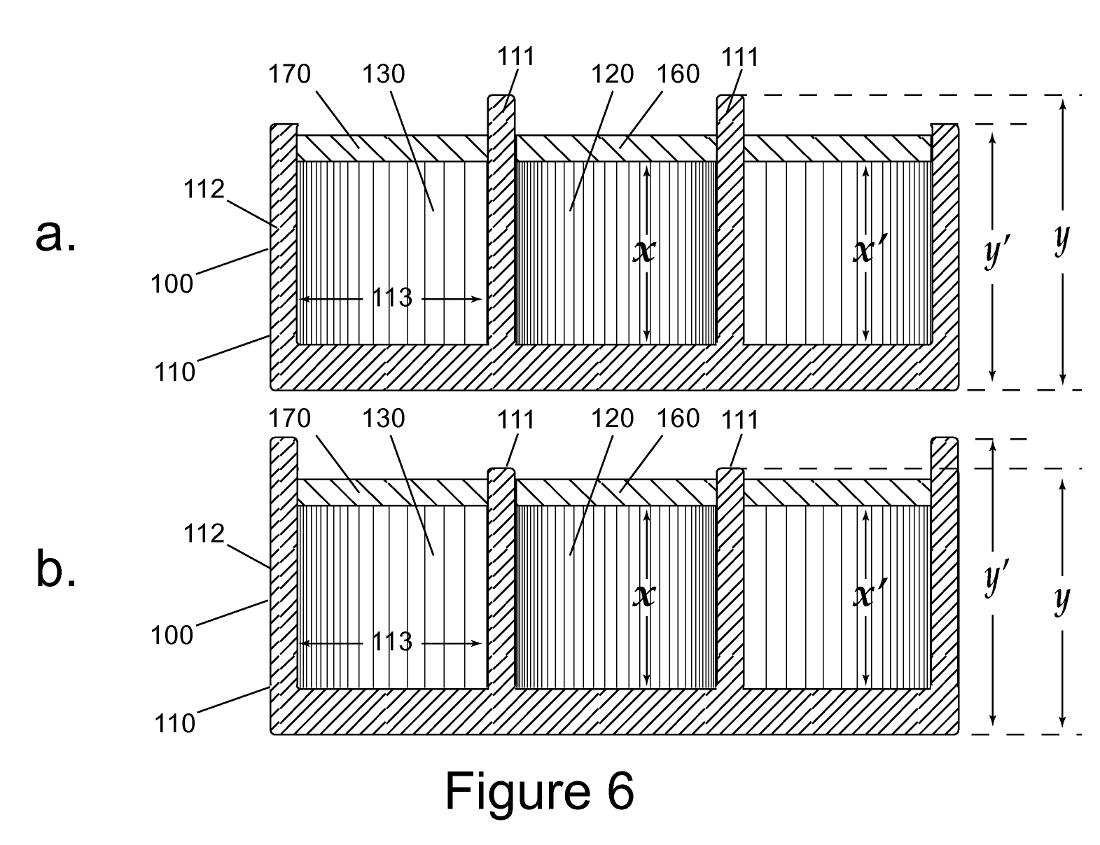

The port 100 will preferably allow caregivers to distinguish between the first and second reservoirs and septa. In certain embodiments, as shown in FIG. 3, the inner wall 111 and optionally the outer wall 112 incorporate radiopaque markings 180, 190 which demarcate the first and second reservoirs when the port 100 is viewed fluoroscopically. The radiopaque markings may be made by applying radiopaque ink to the inner and outer walls 111, 112, or by incorporating a radiopaque material into the inner and outer walls 111, 112. In other embodiments, shown in FIG. 6, the inner wall 111 and the outer wall 112 will each be characterized by a height, y and y′, respectively, which is the distance between the top of the inner or outer wall and the bottom of the housing. In one embodiment, also shown in FIG. 3, the heights of both inner and outer walls 111, 112 will be equal, extending beyond a plane defined by the surfaces of inner septum 160 and outer septum 170, resulting in wall surfaces that demarcate the first and second reservoirs when palpated through the skin, and the inner wall 111 will have a height y that is greater than that of the outer wall 112. In an alternate embodiment, shown in FIG. 6(a) the height y of the inner wall 111 is greater than the height y′ of the outer wall 112. In another embodiment, shown in FIG. 6(b), the height y of the inner wall 111 is less the height y′ of the outer wall 112.

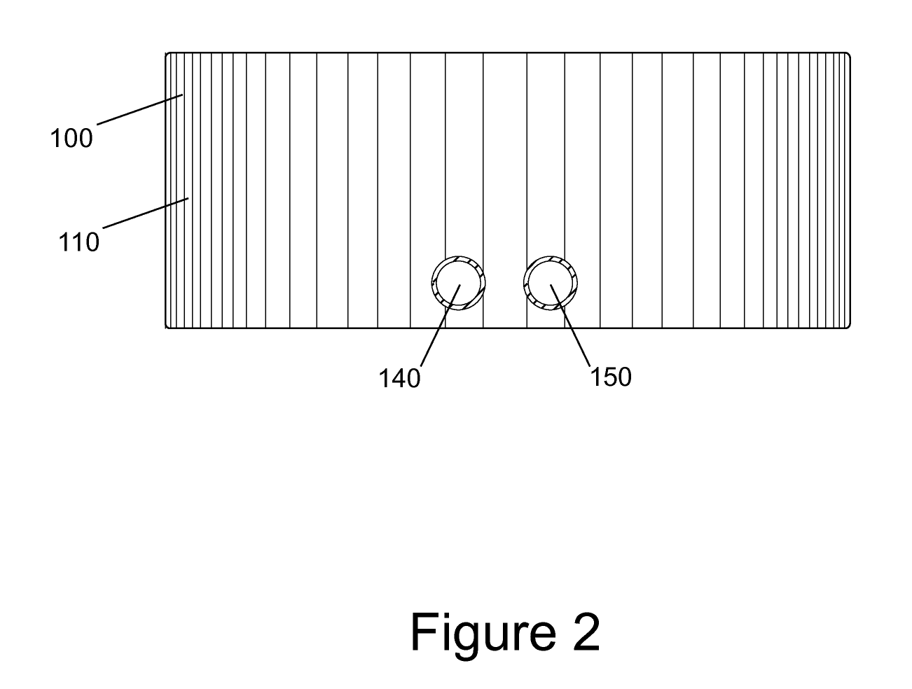

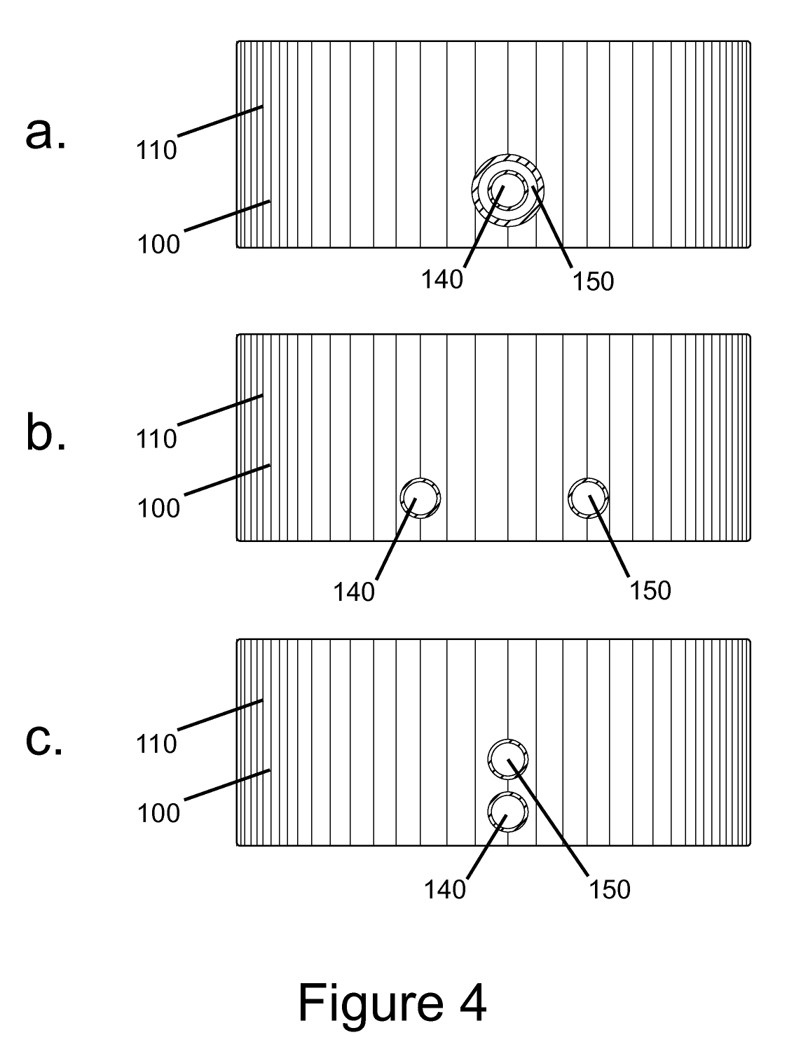

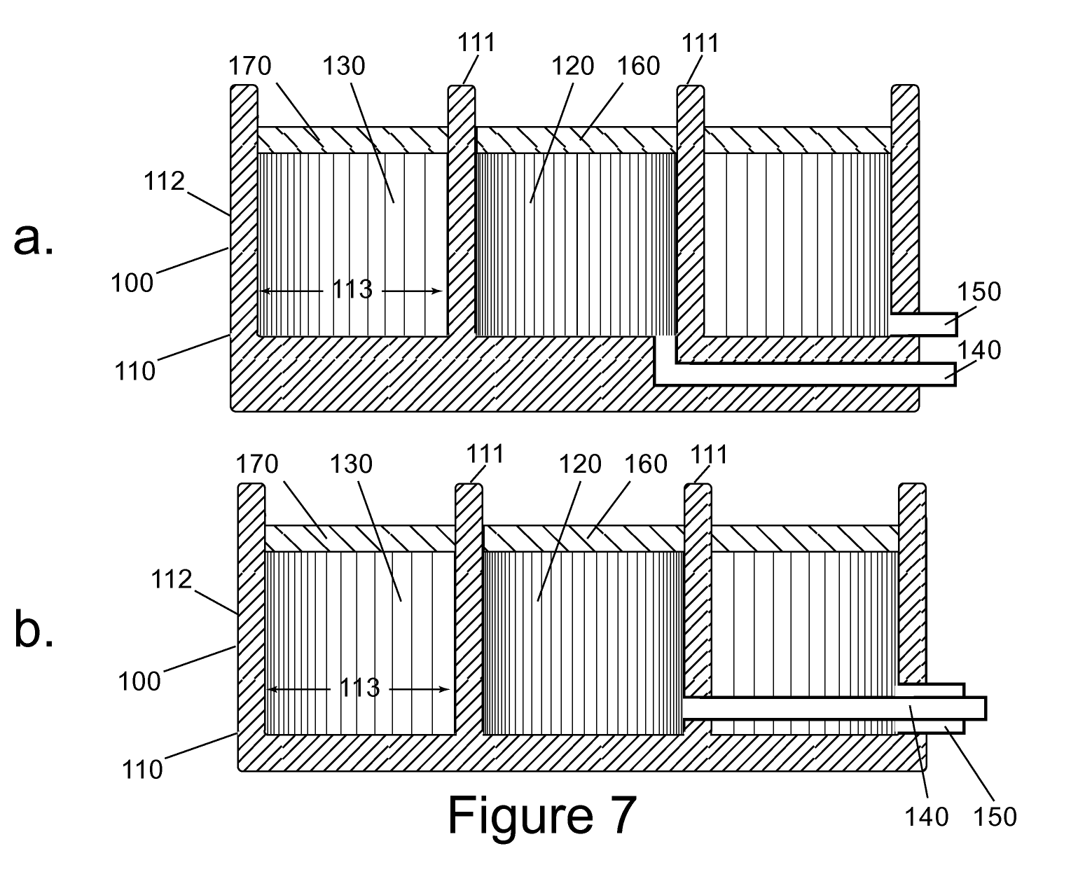

As shown in FIG. 1(b), first and second fluid lines 140, 150 are connected to first and second reservoirs 120, 130 and permit fluid flow between the first the first and second reservoirs and the lumens a catheter or other device that is connected to the port (not shown). Thus, fluid can be introduced into first and second reservoirs 120, 130, and flow through first and second fluid lines 140, 150 into a catheter and then into the body of a patient. The orientation of first and second fluid lines 140, 150 relative to one-another when they exit the housing 110 will determine how the port 100 engages with other devices such as catheters. In the embodiment shown in FIG. 2, the first fluid line 140 extends parallel to the second fluid line 150 to facilitate connection of the port 100 to a multiple-lumen catheter. In another embodiment, shown in FIG. 4(a) and FIG. 7(b), the first fluid line 140 is located, for at least part of its length, inside of the second fluid line 150, so that the first fluid line 140 can be connected to a catheter that runs inside of a catheter attached to the second fluid line 150. In the embodiment shown in FIG. 4(b), the first and second fluid lines 140, 150 will run in different directions so that the port 100 can be attached to a Y connector or other suitable connector. In still another embodiment, shown in FIG. 4(c) and FIG. 7(a), the first fluid line 140 will run underneath the second reservoir 130 and the second fluid line 150.

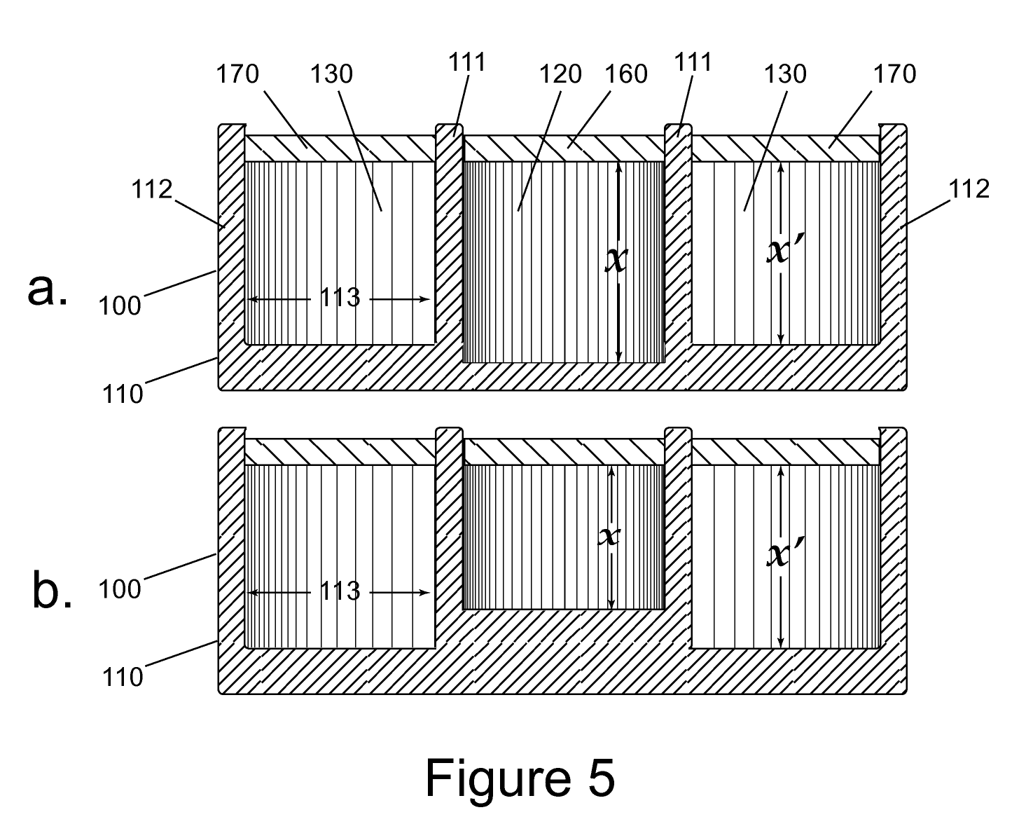

In certain embodiments, the first and second reservoirs 120,130 will each be characterized by a pre-determined depth, which is the distance between the inner surface of the housing 110 and the inner surface of the septum, 160, 170. The depths of the first and second reservoirs 120, 130 can be varied relative to one-another to accommodate different orientations of the first and second fluid lines 140, 150. Preferably, the depths of the first and second reservoirs are sufficient to accommodate a fully-inserted needle. In the preferred embodiment, shown in FIG. 3, the depths x, x′ of the first and second reservoirs 120, 130, respectively, are the same. In the alternate embodiment shown in FIG. 5(a), the first reservoir 120 has a depth x greater than the depth x′ of the second reservoir 130. In the alternate embodiment shown in FIG. 5(b), the first reservoir 120 will have a depth x less than the depth x′ of the second reservoir 130.

One benefit of the current invention, in all of its embodiments, is that the bulk of the port 100 is significantly reduced relative to the current art by arranging the reservoirs one-inside-the-other.

The current invention can be used in any application for which dual-septum ports may be used by those skilled in the art including, but not limited to, high pressure infusion.

Variations, modifications, and other implementations of what is described herein will occur to those of ordinary skill in the art without departing from the spirit and scope of the invention as claimed. Accordingly, the invention is to be defined not by the preceding illustrative description but instead by the spirit and scope of the following claims.

We claim:

1. A medical device comprising:
a housing, having an outer wall and an inner wall, with a space separating said outer wall from said inner wall;
a first reservoir, defined by said inner wall;
a second reservoir, defined by said space separating said outer wall from said inner wall;
a first septum in contact with said inner wall and positioned over said first reservoir;
a second septum in contact with said inner wall and said outer wall and positioned over said second reservoir;
a first fluid line in fluid communication with said first reservoir; and
a second fluid line in fluid communication with said second reservoir.

2. The medical device of claim 1, wherein said outer wall has a first height, and said inner wall has a second height that is greater than said first height.

3. The medical device of claim 1, wherein said outer wall has a first height, and said inner wall has a second height that is less than said first height.

4. The medical device of claim 1, wherein said inner wall and, optionally, said outer wall, comprise radiopaque markings.

5. The medical device of claim 1, wherein the first fluid line is located for part of its length within the second fluid line.

6. The medical device of claim 1, wherein said first reservoir is located concentrically within said second reservoir.

7. The medical device of claim 1, wherein said first reservoir has a first depth, and said second reservoir has a second depth less than the first depth.

8. The medical device of claim 1, wherein said first reservoir has a first depth, and said second reservoir has a second depth greater than or equal to the first depth.

9. A medical device comprising:
a housing having a substantially circular inner wall with a first height located concentrically within a substantially circular outer wall having a second height, and a space separating said inner and outer walls;
a first reservoir defined by said inner wall having a first depth;
a second reservoir defined by said space between said inner and outer walls having a second depth;
a first septum in contact with said inner wall and positioned over said first reservoir;
a second septum in contact with said inner and outer walls and positioned over said second reservoir;
a first fluid line in fluid communication with said first reservoir; and
a second fluid line in fluid communication with said second reservoir.

10. The medical device of claim 9, wherein said first height is greater than said second height.

11. The medical device of claim 9, wherein said first depth is greater than said second depth.

12. The medical device of claim 9, wherein said inner wall and, optionally, said outer wall, comprise radiopaque markings.
